SEQUENCE LISTING (2 sequences)

SEQ ID NO: 1 - DNA, 1542 bp, Homo sapiens
ggatccgccgccactatggcccaagccctgccctggctcctgctgtggatgggcgcgggagtgctgcctgcccacggcacccagcacggcatccgactgccactgcgcagcggactgggaggtgcacctctgggactgcggctgccccgggagaccgacgaagagcccgaggagcccggccggaggggcagctttgtggagatggtggacaacctgaggggcaagtcggggcagggctactacgtggagatgaccgtgggcagccccccgcagacgctcaacatcctggtggatacaggcagcagtaactttgcagtgggtgctgccccccaccccttcctgcatcgctactaccagaggcagctgtccagcacataccgggacctccggaagggtgtgtatgtgccctacacccagggcaagtgggaaggggagctgggcaccgacctggtaagcatcccccatggccccaacgtcactgtgcgtgccaacattgctgccatcactgaatcagacaagttcttcatcaacggctccaactgggaaggcatcctggggctggcctatgctgagattgccaggcctgacgactccctggagcctttctttgactctctggtaaagcagacccacgttcccaacctcttctccctgcagctttgtggtgctggcttccccctcaaccagtctgaagtgctggcctctgtcggagggagcatgatcattggaggtatcgaccactcgctgtacacaggcagtctctggtatacacccatccggcgggagtggtattatgaggtgatcattgtgcgggtggagatcaatggacaggatctgaaaatggactgcaaggagtacaactatgacaagagcattgtggacagtggcaccaccaaccttcgtttgcccaagaaagtgtttgaagctgcagtcaaatccatcaaggcagcctcctccacggagaagttccctgatggtttctggctaggagagcagctggtgtgctggcaagcaggcaccaccccttggaacattttcccagtcatctcactctacctaatgggtgaggttaccaaccagtccttccgcatcaccatccttccgcagcaatacctgcggccagtggaagatgtggccacgtcccaagacgactgttacaagtttgccatctcacagtcatccacgggcactgttatgggagctgttatcatggagggcttctacgttgtctttgatcgggcccgaaaacgaattggctttgctgtcagcgcttgccatgtgcacgatgagttcaggacggcagcggtggaaggcccttttgtcaccttggacatggaagactgtggctacaacattccacagacagatgagtcaaccctcatgaccatagcctatgtcatggctgccatctgcgccctcttcatgctgccactctgcctcatggtgtgtcagtggcgctgcctccgctgcctgcgccagcagcatgatgactttgctgatgacatctccctgctgaagcatcaccatcaccatcactgataa

SEQ ID NO: 2 - DNA, 1628 bp, Homo sapiens
ggatccgccgccactatgggcgcactggcacgagcactgctgctgcctctgctggcacagtggctacttcgagcagctccagagctagctccagctccattcacgctgccactccgagtagctgctgcaacgaaccgcgtagttgcgcccaccccgggacccgggactcctgccgagcgccacgccgacggcttggcgctcgccctggagcctgccctggcgtcccccgcgggcgccgccaacttcttggccatggtagacaacctgcagggggactctggccgcggctactacctggagatgctgatcgggacccccccgcagaagctacagattctcgttgacactggaagcagtaactttgccgtggcaggaaccccgcactcctacatagacacgtactttgacacagagaggtctagcacataccgctccaagggctttgacgtcacagtgaagtacacacaaggaagctggacgggcttcgttggggaagacctcgtcaccatccccaaaggcttcaatacttcttttcttgtcaacattgccactatttttgaatcagagaatttctttttgcctgggattaaatggaatggaatacttggcctagcttatgccacacttgccaagccatcaagttctctggagaccttcttcgactccctggtgacacaagcaaacatccccaacgttttctccatgcagatgtgtggagccggcttgcccgttgctggatctgggaccaacggaggtagtcttgtcttgggtggaattgaaccaagtttgtataaaggagacatctggtatacccctattaaggaagagtggtactaccagatagaaattctgaaattggaaattggaggccaaagccttaatctggactgcagagagtataacgcagacaaggccatcgtggacagtggcaccacgctgctgcgcctgccccagaaggtgtttgatgcggtggtggaagctgtggcccgcgcatctctgattccagaattctctgatggtttctggactgggtcccagctggcgtgctggacgaattcggaaacaccttggtcttacttccctaaaatctccatctacctgagagatgagaactccagcaggtcattccgtatcacaatcctgcctcagctttacattcagcccatgatgggggccggcctgaattatgaatgttaccgattcggcatttccccatccacaaatgcgctggtgatcggtgccacggtgatggagggcttctacgtcatcttcgacagagcccagaagagggtgggcttcgcagcgagcccctgtgcagaaattgcaggtgctgcagtgtctgaaatttccgggcctttctcaacagaggatgtagccagcaactgtgtccccgctcagtctttgagcgagcccattttgtggattgtgtcctatgcgctcatgagcgtctgtggagccatcctccttgtcttaatcgtcctgctgctgctgccgttccggtgtcagcgtcgcccccgtgaccctgaggtcgtcaatgatgagtcctctctggtcagacatcgctggaaatgataactcgaggcatgcggtaccaagcttgtcgagaagtactagaggatcataatcag

(19) United States
(12) Patent Application Publication
(10) Pub. No.: US 2003/0125257 A1
(43) Pub. Date: Jul. 3, 2003

(54) Assay for identifying beta secretase inhibitors

(76) Inventors: Manfred Brockhaus, Bettingen (CH); Heinz Doebeli, Ziefen (CH); Fiona Grueninger, Arlesheim (CH); Philipp Huguenin, Liestal (CH); Eric Argirios Kitas, Aesch (CH); Peter Nelboeck-Hochstetter, Basel (CH)

Correspondence Address: HOFFMANN-LA ROCHE INC., PATENT LAW DEPARTMENT, 340 KINGSLAND STREET, NUTLEY, NJ, 07110

(21) Appl. No.: 10/322,684

(22) Filed: Dec. 18, 2002

(30) Foreign Application Priority Data
Dec. 20, 2001 (EP) 01130282.5

(51) Int. Cl. A61K 38/55; C12Q 1/37; C12N 9/99

(52) U.S. Cl. 514/12; 514/13; 514/14; 514/15; 435/23; 435/184

(57) ABSTRACT

The present invention relates to an assay for identifying inhibitors of beta-secretases comprising the steps of immobilizing a beta-secretase protein on a solid support, contacting it with a test compound in the presence of a tagged beta-secretase inhibitor, and comparing the extent of binding of the tagged beta-secretase inhibitor in the presence and in the absence of the test compound in order to evaluate if the test compound is an inhibitor of beta-secretases. The present invention also relates to a method of screening for inhibitors of beta-secretases, to novel beta-secretase inhibitors and their use as tagged beta-secretase inhibitors for identification of inhibitors of beta-secetase, to a kit for identifying beta-secretase inhibiotrs as well as to novel beta-secretase inhibitors for use in the treatment of Alzheimer's disease and other cerebrovascular amyloidosis.

### FIELD OF THE INVENTION

The present invention relates to an assay for identifying inhibitors of beta-secretases comprising the steps of immobilizing a beta-secretase protein on a solid support, contacting it with a test compound in the presence of a tagged beta-secretase inhibitor, and comparing the extent of binding of the tagged beta-secretase inhibitor in the presence and in the absence of the test compound in order to evaluate if the test compound is an inhibitor of beta-secretases. The present invention also relates to a method of screening for inhibitors of beta-secretases, to novel beta-secretase inhibitors and their use as tagged beta-secretase inhibitors for identification of inhibitors of beta-secretase, to a kit for identifying beta-secretase inhibitors, as well as to novel beta-secretase inhibitors for use in the treatment of Alzheimer's disease and other cerebrovascular amyloidosis.

### BACKGROUND OF THE INVENTION

Alzheimer's disease (AD) is a degenerative brain disorder characterized clinically by progressive loss of memory, temporal and local orientation, cognition, reasoning, judgment and emotional stability. AD is a common cause of progressive dementia in humans and is one of the major causes of death in the United States. AD has been observed in all races and ethnic groups worldwide, and is a major present and future health problem. No treatment that effectively prevents AD or reverses the clinical symptoms and underlying pathophysiology is currently available (for review see Annu Rev Cell Biol., 1994, 10, 373-403).

Histopathological examination of brain tissue derived upon autopsy or from neurosurgical specimens in effected individuals revealed the occurrence of amyloid plaques and neurofibrillar tangles in the cerebral cortex of such patients. Similar alterations were observed in patients with Trisomy 21 (Down's syndrome), and hereditary cerebral hemorrhage with amyloidosis of the Dutch-type.

Neurofibrillar tangles are nonmembrane-bound bundles of abnormal proteinaceous filaments and biochemical and immunochemical studies led to the conclusion that their principle protein subunit is an altered phosphorylated form of the tau protein (reviewed in Annu Rev Neurosci., 1994, 17, 489-517).

Biochemical and immunological studies revealed that the dominant proteinaceous component of the amyloid plaque is an approximately 4.2 kilodalton (kD) protein of about 39 to 43 amino acids. This protein was designated A-beta-amyloid peptide, and sometimes beta/A4; referred to herein as A-beta. In addition to deposition of A-beta in amyloid plaques, A-beta is also found in the walls of meningeal and parenchymal arterioles, small arteries, capillaries, and sometimes, venules. A-beta was first purified, and a partial amino acid reported, in 1984 (Biochem. Biophys. Res. Commun., 1984, 120, 885-890). The isolation and sequence data for the first 28 amino acids are described in U. S. Pat. No. 4,666,829.

Compelling evidence accumulated during the last decade revealed that A-beta is an internal polypeptide derived from a type 1 integral membrane protein, termed beta amyloid precursor protein (APP). APP is normally produced by many cells both in vivo and in cultured cells, derived from various animals and humans. A-beta is derived from cleavage of APP by an enzyme (protease) system(s), collectively termed secretases.

The existence of at least four proteolytic activities has been postulated. They include beta-secretase(s), generating the N-terminus of A-beta, alpha-secretase(s) cleaving around the 16/17 peptide bond in A-beta, and gamma-secretases, generating C-terminal A-beta fragments ending at position 38, 39, 40, 42, and 43 or generating C-terminal extended precursors which are subsequently truncated to the above polypeptides.

Several lines of evidence suggest that abnormal accumulation of A-beta plays a key role in the pathogenesis of AD. Firstly, A-beta is the major protein found in amyloid plaques. Secondly, A-beta is neurotoxic and may be causally related to neuronal death observed in AD patients. Thirdly, missense DNA mutations at position 717 in the 770 isoform of APP can be found in affected members but not unaffected members of several families with a genetically determined (familial) form of AD. In addition, several other APP mutations have been described in familial forms of AD. Fourthly, similar neuropathological changes have been observed in transgenic animals overexpressing mutant forms of human APP. Fifthly, individuals with Down's syndrome have an increased gene dosage of APP and develop early-onset AD.

Taken together, these observations strongly suggest that A-beta depositions may be causally related to the AD.

It is hypothesized that inhibiting the production of A-beta will prevent and reduce neurological degeneration, by controlling the formation of amyloid plaques, reducing neurotoxicity and, generally, mediating the pathology associated with A-beta production. One method of treatment would therefore be based on drugs that inhibit the formation of A-beta in vivo.

Methods of treatment could target the formation of A-beta through the enzymes involved in the proteolytic processing of APP. Compounds that inhibit beta- or gamma-secretase activity, either directly or indirectly, could control the production of A-beta.

Beta-secretase has been descibed in several publications including EP publication number EP0855444, and PCT publication numbers WO0017369, WO0058479, WO0047618, WO0100663 and WO0100665.

Two genes, BACE (synonyms are Asp-2, Memapsin-2) and BACE-2 (synonyms are Asp-1, Memapsin-1), code for beta-secretases which belong to the family of membrane-spanning aspartate proteases. They are believed to be the first enzymes in the cascade of APP degradation leading to A-beta peptide production, which is responsible for amyloid deposition in the brain of AD patients. Conceptually, the inhibition of beta-secretase would protect from AD by diverting the amyloidogenic pathway of APP degradation to the non-amyloidogenic pathway via the alpha-secretase cleavage of APP.

BACE−/−mice were found to be viable in spite of the ubiquitous expression of BACE in most tissues, and its high expression in brain and pancreas (Nature Neurosci., 2001, 4, 231-232). The finding that there are no apparent adverse effects associated with BACE deficiency in mice suggests that inhibition of BACE in humans may not have mechanism-based toxicity, a problem which is intensely debated for gamma-secretase, which controls vital Notch signaling.

Cellular screening methods for inhibitors of A-beta production, testing methods for the in vivo suppression of A-beta production, and assays with membranes or cellular extracts for the detection of secretase activity are known in the art and have been disclosed in numerous publications, including PCT publication number WO 98/22493, and U.S. Pat. Nos. 5,703,129 and 5,593,846; all hereby incorporated by reference.

The standard assay for beta-secretase is a fluorescence assay which is based on the cleavage of a peptide substrate carrying a fluorophor and a quencer. When used for screening purposes this assay is prone to “false positive hits” either because the compounds show self-fluorescence or are strongly coulored, or because they absorb the enzyme and precipitate from solution. Therefore, an alternative assay is of great value for beta-secretase inhibitor screening.

Surprisingly, it was found that by labelling transition-state mimetics of beta-secretases with a sufficiently high radioactivity they can be used as direct probe for active site binding to beta-secretase.

The present invention relates to an assay for identifying inhibitors of beta-secretases comprising the steps of immobilizing a beta-secretase protein on a solid support, contacting it with a test compound in the presence of a tagged beta-secretase inhibitor, and comparing the extent of binding of the tagged beta-secretase inhibitor in the presence and in the absence of the test compound in order to evaluate if the test compound is an inhibiotr of beta-secretases. Moreover, the present invention relates to a method of screening for inhibitors of beta-secretases, to novel beta-secretase inhibitors and their use as tagged beta-secretase inhibitors for identification of inhibitors of beta-secretase, to a kit for identifying beta-secretase inhibitors, as well as to novel beta-secretase inhibitors for use in the treatment of Alzheimer's disease and other cerebrovascular amyloidosis.

### SUMMARY OF THE INVENTION

This invention is directed to a beta-secretase inhibitor of formula Y-P4-P3-P2-P1-P1′-P2′-P3′-P4′-W, wherein P1 is defined as Leustatin, Chastatin or Tyrstatin; P2 is defined as Asn; P3 is defined as Val, Cpe, Che or Cha; P4 is defined as Glu; P1′ is defined as Val; P2′ is defined as Ala; P3′ is defined as Glu; P4′ is defined as Tyr, Cha, Phe(I) or Tyr(I2); Y is defined as 0 to 10 amino acid residues; and W is defined as 0 to 10 amino acid residues; or a combination thereof.

In another embodiment, this invention is directed to an assay for identifying beta-secretase inhibitors comprising the steps of (a) immobilizing beta-secretase protein; (b) contacting the immobilized beta-secretase protein with a test compound followed by a tagged beta-secretase inhibitor; (c) incubating the assay components; and (d) measuring bound tagged beta-secretase inhibitor.

In yet another embodiment, this invention is directed to a method of screening for compounds capable of inhibiting a beta-secretase activity comprising measuring the binding activities of a tagged beta-secretase inhibitor to a beta-secretase in the presence of a test compound and determining the level of test compound competing with the tagged beta-secretase inhibitor for active-site binding based on the binding activity.

In yet another embodiment, this invention is directed to a pharmaceutical composition comprising a pharmaceutically acceptable carrier and a therapeutically effective amount of a beta-secretase inhibitor, particularly according to Formula 1.

In yet another embodiment, this invention is directed to a method of treating a patient afflicted or having a predisposition for cerebrovascular amyloidosis comprising administering to the patient a pharmaceutically effective dose of a compound comprising a beta-secretase inhibitor, preferably according to Formula I. The cerebrovascular amyloidosis includes Alzheimer's disease.

### BRIEF DESCRIPTION OF THE DRAWING

FIG. 1: Formula of building blocks of Formula (I)

FIG. 2: Formula of tritiated building blocks of Formula (I), wherein the tritium is depicted as T.

FIG. 3: The graph shows the inhibition curves generated by published peptidomimetic inhibitors (peptide H-4848 (Bachem) described in Nature, 1999, 402, 537 and peptide H-5108 (Bachem) described in J. Am. Chem. Soc., 2000, 122, 3522) and other active site directed inhibitors. It also shows that the assay is not prone to strongly coulored compounds (Congo red) or the “sticky compounds” which were previously scored as false positive in the FRET assay.

FIG. 4: Equilibrium binding of tritiated compound A to purified BACE as decribed in example 6 (A). The Scatchart analysis reveals a single binding site isotherm and competitive inhibition by the peptide H-4848 (B).

FIG. 5: Evaluation of peptidomimetic compounds as beta-secretase inhibitors in the assay of the present invention over a broad range of IC-50 values (A). Correlation of IC-50 values for 19 peptidomimetic BACE inhibitors derived by the FRET assay and the competitive radioligand binding assay (B).

FIG. 6: Effect of BSA on binding inhibition illustrated in a Scatter plot (% control dpm in plate (P) and column (C) pooling cocktail plates (3200 data points)). The result illustrates the heterogeneity of binding inhibition observed without BSA in the binding buffer. Ideally, each compound which is represented by one dot has to give identical inhibition in the column pool and in the plate pool, and be scattered along the diagonal line (A). Binding inhibition without BSA shown in a Scatter plot (% control dpm in plate (P) and column (C) pooling cocktail plates (3200 data points)). This result illustrates the homogeneity of binding inhibition observed with BSA in the binding buffer (B).

FIG. 7: Dose-dependent inhibition of radioligand binding by compound H-4848 (Bachem, described in Nature, 1999, 402, 537) under the influence of 0.02% cholic acid (full circles), 0.02% Tween 20 (Open circles), and 0.1% bovine serum albumin (BSA, full triangles).

### DETAILED DESCRIPTION OF THE INVENTION

The present invention relates to an assay for identifying inhibitors of beta-secretases comprising the steps of immobilizing a beta-secretase protein on a solid support, contacting it with a test compound in the presence of a tagged beta-secretase inhibitor, and comparing the extent of binding of the tagged beta-secretase inhibitor in the presence and in the absence of the test compound in order to evaluate if the test compound is an inhibitor of beta-secretases. Moreover, the present invention relates to a method of screening for inhibitors of beta-secretases, to novel beta-secretase inhibitors and their use as tagged beta-secretase inhibitors for identification of inhibitors of beta-secretase, to a kit for identifying beta-secretase inhibitors, as well as to novel beta-secretase inhibitors for use in the treatment of Alzheimer's disease and other cerebrovascular amyloidosis.

The present invention provides an assay for identifying beta-secretase inhibitors comprising the steps of immobilizing beta-secretase protein on a solid support, adding a test compound and subsequently a tagged beta-secretase inhibitor, incubating the assay components for equilibrium binding, and measuring bound tagged beta-secretase inhibitor.

As used herein, “beta-secretase” is defined as an aspartyl-protease generating the N-terminus of A-beta. Preferred beta-secretases are human BACE and BACE-2. Most preferred are human BACE and BACE-2 which are encoded by the nucleotide sequences as disclosed in SEQ ID NO. 1 and SEQ ID NO. 2, respectively. The beta-secretase can be a full-length beta-secretase or a truncated beta-secretase at least exhibiting the active-site. Preferably, beta-secretase is a full-length beta-secretase. Beta-secretases may contain amino acid substitutions if such substitutions do not generally alter the beta-secretase activity. Amino acid substitutions in proteins and polypeptides which do not essentially alter biological activity are known in the art and are described by H. Neurath and R. L. Hill in “The Proteins”, Academic Press, New York (1979). Six general classes of amino acid side chains, categorized as described above, include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). Beta-secretases can additionally contain sequences of several amino acids which are encoded for by “linker” sequences. These sequences arise as a result from the expression vectors used for recombinant expression of beta-secretases. Beta-secretases of the present invention can also contain specific sequences attached to the N- or the C-terminus that preferably bind to an affinity carrier material. Examples of such sequences are sequences containing at least two adjacent histidine residues (see European Patent No. EP282042). Such sequences bind selectively to nitrilotriacetic acid nickel chelate resins. Beta-secretases, which contain such a specific sequence, can therefore be separated selectively from the remaining polypeptides or attached to a solid support for immobilization. The cDNA sequence of the beta-secretase BACE coding for six C-terminal His residues is shown in SEQ ID. NO. 1.

Natural or recombinantly produced beta-secretase can be used in this assay. A “recombinant protein” is a protein isolated, purified, or identified by virtue of expression in a heterologous cell, the cell having been transduced or transfected, either transiently or stably, with a recombinant expression vector engineered to drive expression of the protein in the host cell. Recombinant beta-secretase can be produced in procaryotic cells, e.g., E. coli, in yeast, e.g., S. pombe or in eukaryotic cells, e.g. HEK 293, Sf9 insect cells. Preferably, Sf9 insect cells are used for high expression of recombinant beta-secretase. The beta-secretase used in the assay may be purified. The term “purified” as used herein refers to polypeptides, that are removed from their natural environment or from the source of recombinant production, isolated or separated, and are at least 60% and more preferably at least 80% free from other components, e.g., membranes and microsomes, with which they are naturally associated. The foregoing notwithstanding, such a descriptor does not preclude the presence in the same sample of splice- or other protein variants (glycosylation variants) in the same, otherwise homogeneous, sample.

A protein or polypeptide is generally considered to be “substantially purified” if a sample containing it shows a main protein band on a Commassie-stained acrylamide electrophoretic gel.

The term “immobilizing” as used herein can be a direct immobilizing of beta-secretase on a solid support or an indirect immobilizing of beta-secretase via an attached linker or with the use of an additional linker. The attached linker may be histidine residues or the linker may be streptavidin or an antibody, preferably an antibody directed to beta-secretase. The solid support may be microplates or beads. Preferably, the microplates used in this assay can be white or black, preferred are white Optiplates (Optiplate Packard). The coating reaction is carried out with a protein concentration of 1 μg/ml to 50 μg/ml, preferably of 10 μg/ml. For the coating reaction a buffer is used adjusted to a pH-range of 3 to 8, preferably a pH-range of 5 to 6. Most preferred is a citrate buffer adjusted to pH 5.5.

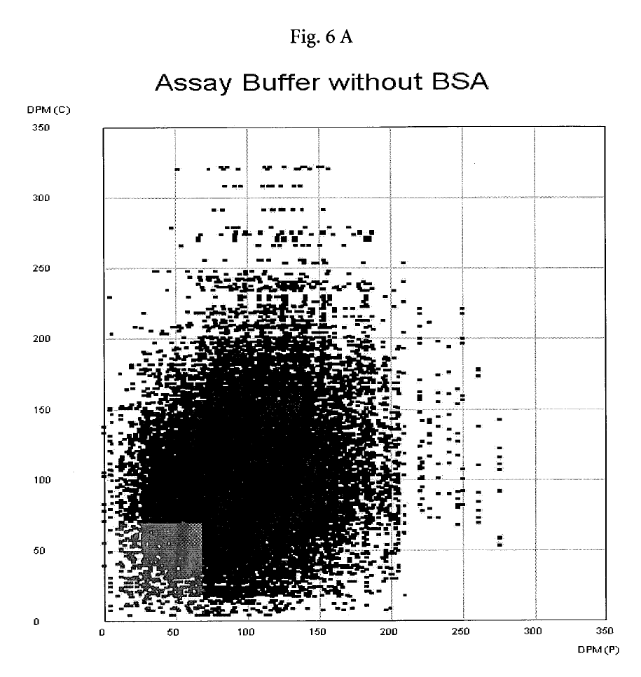

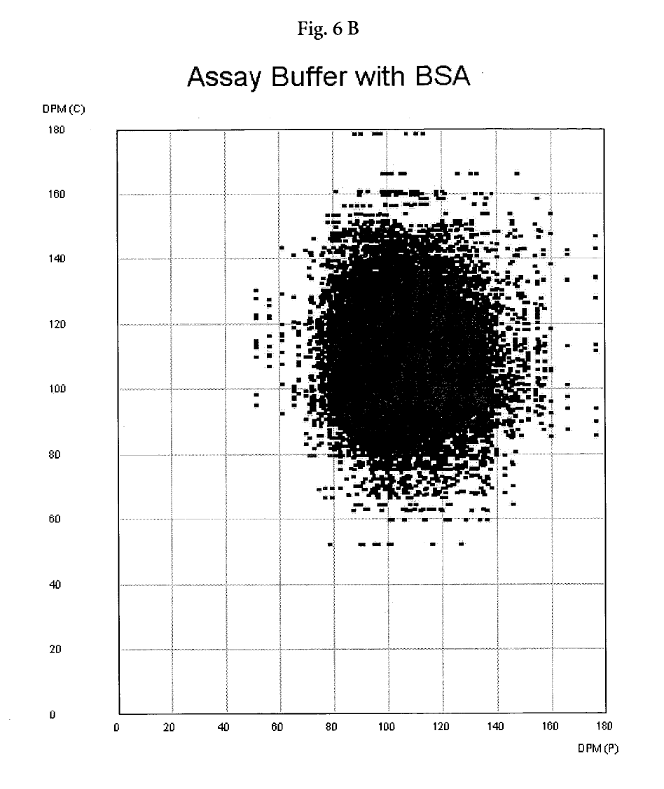

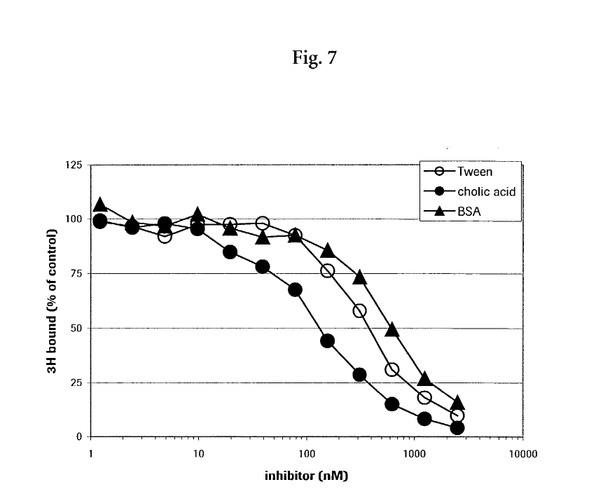

The binding buffer in which the binding assay is carried out may be a buffer adjusted to a pH-range of 2.5 to 6. Preferably, the buffer is a citrate or an acetate buffer adjusted to a pH of 3.5 to 4.5. Most preferred is a citrate buffer with the pH of 4.1. The binding buffer may contain a high molecular weight protein whose presence prevents non-specific binding. Preferably, the protein is BSA (FIGS. 6A and B). Most preferably, the concentration of BSA in the binding buffer is 0.1% w/v. The binding buffer may also contain a non-ionic detergent or an ionic detergent. Preferably, the non-ionic detergent is Tween-20. Preferably, the ionic detergent is cholic acid or deoxycholic acid. More preferably, the ionic detergent is cholic acid. Most preferably, the concentration of the non-ionic or of the ionic detergent in the binding buffer is 0.02%. The presence of cholic acid in the binding buffer could further reduce the IC-50 value of a test substance by 3 to 4 times compared to the presence of Tween-20 (FIG. 7). The components of the assay are incubated until the adjustment of equilibrium binding from 10 minutes to 24 hours at room temperature or at 4 ° C. Preferably, the assay compounds are incubated at room temperature for 1.5 hours.

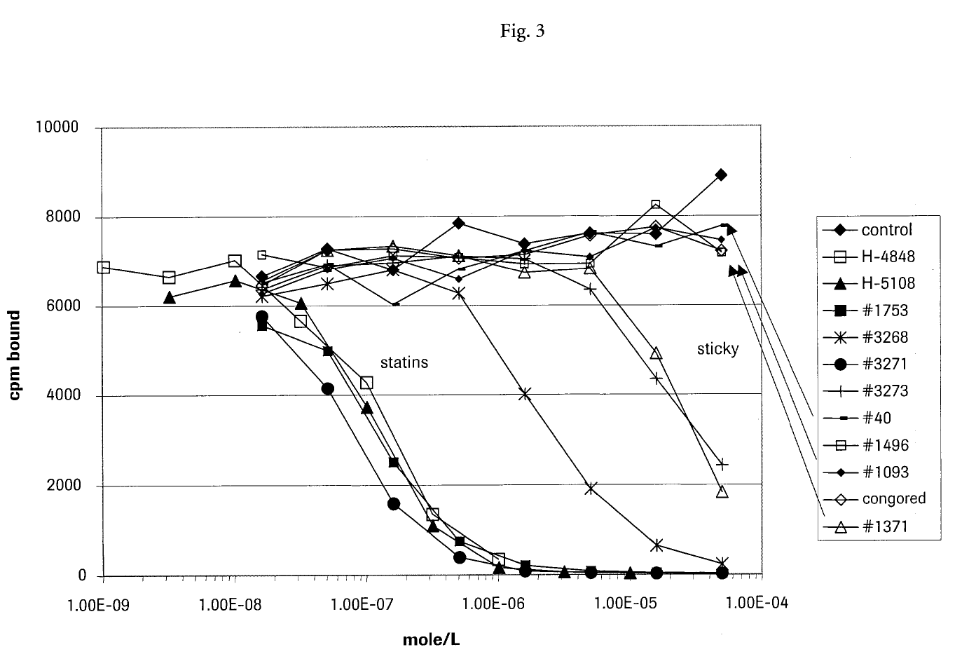

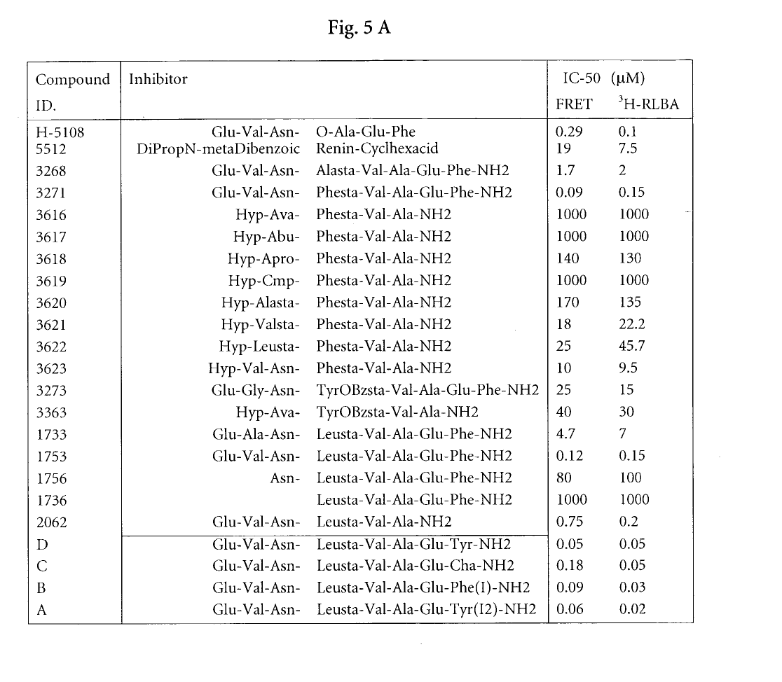

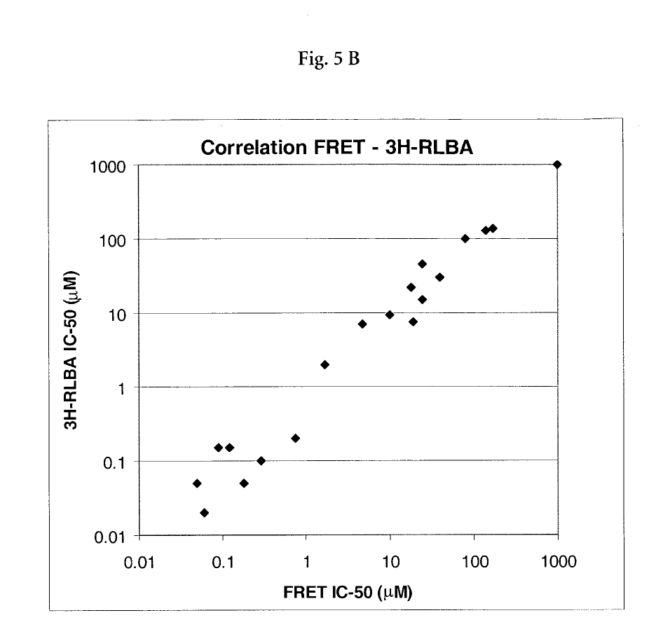

As used herein, “beta-secretase inhibitor” is intended to mean any compound which specifically binds to the active-site of beta-secretase and thereby inhibits the cleavage of a natural beta-secretase substrate, e.g., APP. The present invention relates to competitive binding studies for the screening for inhibitors specific for beta-secretase active site. When comparing the results obtained with test compounds in the competition assay of the present invention and in a FRET assay (FIGS. 5A and B) it could be shown that test compounds inhibiting the binding of a tagged beta-secretase to beta-secretase also inhibit the proteolytic activity of beta-secretase leading to production of A-beta over a broad range of IC-50 values. Thus, the binding of tagged beta-secretase inhibitors to isolated beta-secretase is useful in the identification of inhibitors of A-beta production through competitive binding assays. Moreover, it could be shown that competitive binding assays with labelled beta-secretase inhibitors are not prone to strongly coulored compounds (Congo red) or to “sticky compounds” which were previously scored as false positive in other assay formats (FIG. 3).

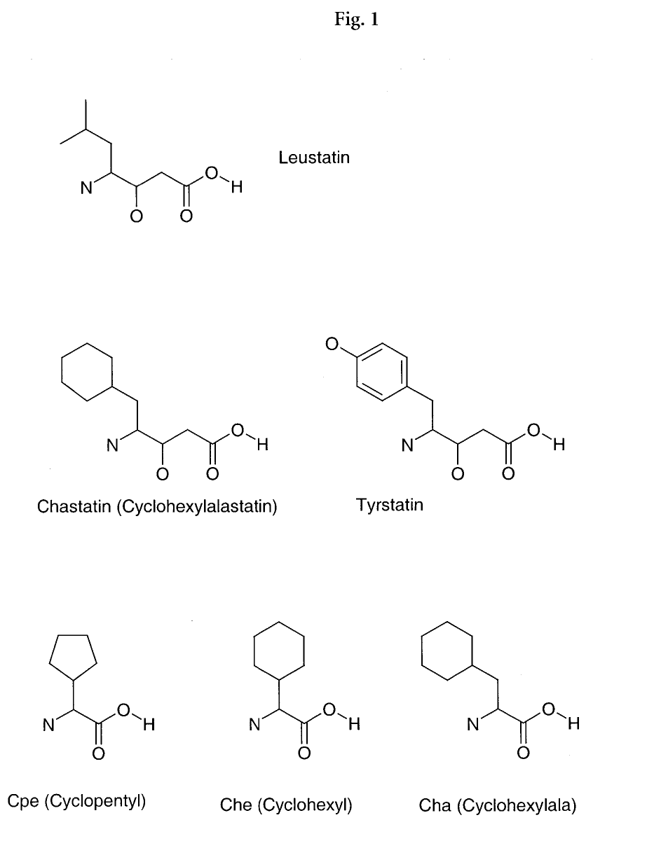

The present invention also relates to novel beta-secretase inhibitors which are peptidomimetic compounds based on a non-cleavable transition state mimetic. The beta-secretase inhibitors of the present invention are peptidomimetics of the Formula (I): Y-P4-P3-P2-P1-P1′-P2′-P3′-P4′-W; wherein P1 is defined as Leustatin, Chastatin or Tyrstatin; P2 is defined as Asn; P3 is defined as Val, Cpe, Che or Cha; P4 is defined as Glu; P1′ is defined as Val; P2′ is defined as Ala; P3′ is defined as Glu; P4′ is defined as Tyr, Cha, Phe(I) or Tyr(I2); Y is defined as 0 to 10 amino acid residues; and W is defined as 0 to 10 amino acid residues; as well as any combinations thereof. Preferably, Y contains no amino acids. Preferably, W contains no amino acids. The formulas of Leustatin, Chastatin, Tyrstatin, Cpe, Che and Cha are defined in FIG. 1.

Beta-secretase inhibitors used in the assay of the present invention are shown in FIG. 5A. Specific examples of Formula (I) are compounds A, B, C and D exhibited in the bottom part of the table of Fig.

The transition state analogs within the peptides are arranged one beneath the other. O is defined as in Science 290, 2000, 150-153. Hyp is defined as hydroxyprolin, Ava is defined as δ-aminovaleric acid, Abu is defined as γ-aminobutyric acid, Apro is defined as β-aminopropionic acid, Cmp is defined as carboxymethylpiperidine and TyrOBzsta is defined as tyrosyl-O-benzylstatin.

The peptidomimetic beta-secretase inhibitors can be chemically synthesized using standard methods known in the art, preferably solid state methods, such as the methods of Merrifield (J Am. Chem. Soc., 1963, 85, 2149-2154) and of Atherton and Sheppard, Solid Phase Peptide Synthesis: A Practical Approach (IRL Press Oxford 1989).

The present invention also relates to beta-secretase inhibitors of Formula (I) for the preparation of tagged beta-secretase inhibitors and to beta-secretase inhibitors of Formula (I) which are tagged.

As used herein, “tagged beta-secretase inhibitor”, is intended to mean “beta-secretase inhibitor” compounds which are tagged. By “tagged” or “tagged beta-secretase inhibitor”, it is meant that the subject inhibitor compounds contain a tag which is suitable for detection in an assay system or upon administration to a mammal. Suitable tags are known to those skilled in the art and include, for example, radioisotopes, fluorescent groups, biotin (in conjunction with streptavidin complexation), and photoaffinity groups. Suitable radioisotopes are known to those skilled in the art and include, for example, isotopes of halogens (such as chlorine, fluorine, bromine and iodine), and metals including technetium and indium. Preferred radioisotopes include ³H, ¹¹C, ¹⁸F, ³²p, ³³p, ³⁵S, ¹²³I, ¹²⁵I, and ¹³¹I. Most preferred are ³H, ¹²⁵I and ¹³¹I. Radiolabelled compounds of the invention may be prepared using standard radiolabelling procedures well known to those skilled in the art. Suitable synthesis methodology is described in detail below. The beta-secretase inhibitors of the invention may be radiolabelled either directly (that is, by incorporating the radiolabel directly into the compounds) or indirectly (that is, by incorporating the radiolabel into the compounds through a chelating agent, where the chelating agent has been incorporated into the compounds). Also, the radiolabelling may be isotopic or nonisotopic. With isotopic radiolabelling, one atom or a group of atoms already present in the compounds of the invention is substituted with (exchanged for) the radioisotope.

With nonisotopic radiolabelling, the radioisotope is added to the compounds without substituting with (exchanging for) an already existing group. Direct and indirect radiolabelled compounds, as well as isotopic and nonisotopic radiolabelled compounds are included within the phrase “radiolabelled beta-secretase inhibitors” as used in connection with the present invention.

Such radiolabeling should also be reasonably stable, both chemically and metabolically, applying recognized standards in the art. Also, although the compounds of the invention may be labelled in a variety of fashions with a variety of different radioisotopes, as those skilled in the art will recognize, such radiolabelling should be carried out in a manner such that the high binding affinity and specificity of the unlabelled or untagged beta-secretase inhibitor to the beta-secretase is not significantly affected. By not significantly affected, it is meant that the binding affinity and specificity is not affected more than about 3 log units, preferably not more than about 2 log units, more preferably not more than about 1 log unit, even more preferably not more than about 500%, and still even more preferably not more than about 250%, and most preferably the binding affinity and specificity is not affected at all.

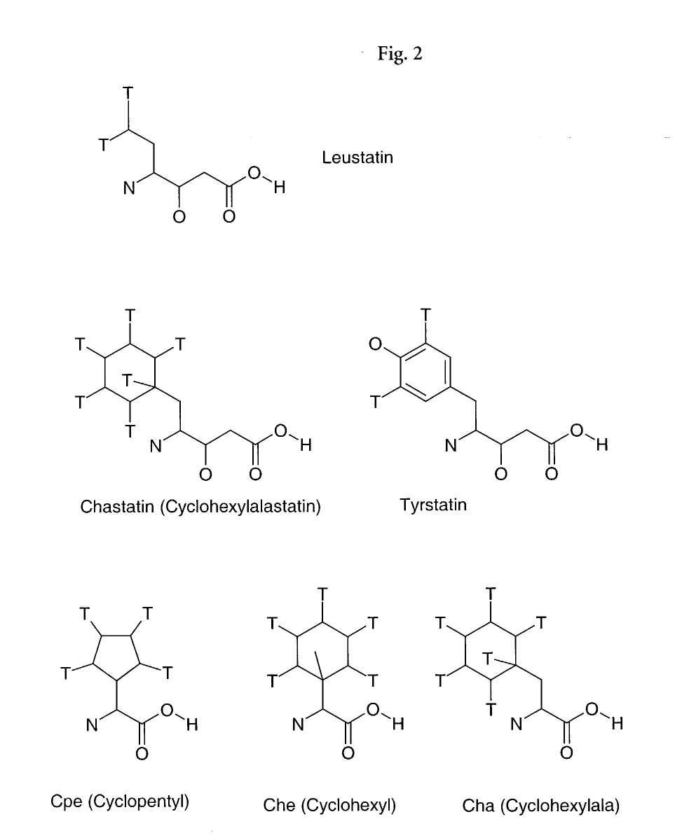

For radiolabelled beta-secretase inhibitors, the label may appear at any position on the beta-secretase inhibitor and it may have one, two or more radioactive isotopes integrated in its structure. Preferred radiolabelled compounds of the invention are beta-secretase inhibitors radiolabelled with tritium. More preferred radiolabelled compounds of the invention are radiolabelled compounds of Formula (I) wherein there are one, two or more radioactive isotopes integrated in its structure and wherein the radiolabel is located on P1, P3 and/or P4′. Most preferred are beta-secretase inhibitors of Formula (I) wherein the radiolabel on P1, P3 and/or P4′ is ³H or ¹²³I, ¹²⁵I, or ¹³¹I. FIG. 2 shows tritiated building blocks of Formula (I) which may be integrated at positions P1, P3 and P4′.

As described in Example 4, a beta-secretase inhibitor of Formula (I) with di-iodotyrosine at position P4′ (Compound A) may be chosen for radiolabelling because the iodine can be exchanged for ³H by reduction on palladium. The reduction yields a compound which is chemically indistinguishable from Compound A.

The radiolabelled beta-secretase inhibitor may have a specific activity in the range of 500 mCi/mmole to 60 Ci/mmole. Preferably, it has a specific activity of 55 Ci/mmole. The bound radiolabeled beta-secretase inhibitor may be measured by addition of a scintillator. Preferably, the scintillator is Microscint20 or Microscint40 (Packard).

Alternatively, it is well known in the art that a scintillation proximity assay (SPA) could be employed in the radioligand competition binding assay of the invention. For example, purified proteins can be immobilized onto the SPA support, after which the support is then incubated with a tagged beta-secretase inhibitor in the presence of a test compound. The SPA support, by nature of its construction, magnifies the radioactive scintillation signal of bound radioactive compounds while not magnifying the radioactive signal of radioactive compounds free in solution. Therefore, the bound tagged beta-secretase inhibitor is detected and quantified by scintillation counting in the presence of free tagged beta-secretase inhibitor.

It is understood that the process of separating bound tagged beta-secretase inhibitor from free tagged beta-secretase inhibitor can be conducted in a number of methods. For example, the process of separating includes, but is not limited to, washing, filtration or centrifugation. The process of separating is intended to facilitate quantification of bound tagged beta-secretase inhibitor. Therefore, the process of separating is also intended to encompass homogeneous techniques, for example SPA, where free tagged beta-secretase inhibitor in situ is not separated from the bound tagged beta-secretase inhibitor.

In the present invention, it has been discovered that the radiolabelled compounds above are useful as beta-secretase inhibitors and thus the radiolabeled compounds of the invention may also be employed for therapeutic purposes and the purpose of radioimaging (Q J Nucl. Med., 1997, 41(2), 163-169) and PET imaging (Clin. Geriatr. Med., 2001, 17(2), 255-279).

As used herein, “test compound” is intended to mean any compound which is being screened for inhibiting the binding of the tagged beta-secretase inhibitor to beta-secretase and therefore inhibit the production of A-beta, using the assay of the invention described herein. It is understood that a “test compound”, which is active in the assay of the invention for inhibiting binding to BACE, can subsequently be used in the assay of the invention as a “tagged beta-secretase inhibitor”, as defined above, once the compound has been tagged. It is also understood that a “test compound”, which is active in the assay of the invention for inhibiting binding to BACE, can subsequently be used in pharmaceutical compositions for the treatment of degenerative neurological disorders involving A-beta production, preferably for the treatment of AD.

As used herein, “bound tagged beta-secretase inhibitor” is intended to mean total binding of tagged beta-secretase inhibitor including specific and non-specific binding. Non-specific binding is assessed by competition with a saturation concentration of another known beta-secretase inhibitor. Specific binding of tagged beta-secretase inhibitor is then determined by subtracting the non-specific binding from the total binding of the tagged beta-secretase inhibitor.

As used herein, “inhibitory concentration” is intended to mean the concentration at which the “potential inhibitor of beta-secretase” compound screened in the assay of the invention displaces 50% of a tagged inhibitor. Examples of “inhibitory concentration” values range from IC-50 to IC-90, and are preferably, IC-50, IC-60, IC-70, IC-80 or IC-90, which represent 50%, 60%, 70%, 80% and 90% displacement of the tagged inhibitor, respectively. More preferably, the “inhibitory concentration” is measured as the IC-50 value. It is understood that a designation for IC-50 is the half maximal inhibitory concentration. The IC-50 of a tagged beta-secretase inhibitor used in the assay and the method of the present invention may be ≦5 μM. More preferred, the IC-50 is ≦1 μM. Most preferred, the IC-50 is ≦0.25 μM.

The present invention relates also to the assay as described above, wherein the tagged beta-secretase inhibitor has the Formula (I).

A further embodiment of the present invention are tagged beta-secretase inhibitors of Formula (I) for use in the assay of the invention as described above.

Moreover, the present invention relates to the use of a tagged beta-secretase inhibitor for the identification of inhibitor compounds of beta-secretases. For this use, the tagged beta-secretase inhibitor may have the Formula (I) and it may include a radioactive tag or, more specifically, a tritium tag.

The present invention further relates to a method of screening for compounds capable of inhibiting a beta-secretase activity comprising measuring binding of a tagged beta-secretase inhibitor to a beta-secretase in the presence of a test compound and determining if the test compound could compete with the tagged beta-secretase inhibitor for active-site binding.

The present invention also relates to the method as descibed above, wherein the tagged beta-secretase inhibitor has the Formula (I).

Moreover, the present invention relates to tagged beta-secretase inhibitors for use in the method as described above.

In a further embodiment, the present invention relates to a kit for identifying a beta-secretase inhibitor comprising natural or recombinantly produced beta-secretase polypeptide and a tagged beta-secretase inhibitor.

In a further embodiment, the present invention relates to a kit comprising the components necessary for carrying out the assay or the method of the present invention selected from the group of a solid support for immobilizing beta-secretase protein, beta-secretase protein, coating buffer, tagged beta-secretase inhibitor, and binding buffer.

Moreover, the present invention relates to novel inhibitors of beta-secretases identified by the assay and the method of the present invention. These could then be used themselves for identifying novel inhibitors of beta-secretase.

The present invention further provides a pharmaceutical composition comprising a pharmaceutically acceptable carrier and a therapeutically effective amount of an inhibitor of beta-secretases identified by the assay and the method of the present invention; as well as pharmaceutically acceptable salts thereof.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, benzenesulfonic, toluenesulfonic, methanesulfonic, ethane disulfonic, oxalic, isethionic, and the like.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference.

“Stable compound” and “stable structure” are meant to indicate a compound that is sufficiently robust to survive isolation to a useful degree of purity from a reaction mixture, and formulation into an efficacious therapeutic agent.

Moreover, the present invention relates to the use of such beta-secretase inhibitor compounds identified by the assay and the method of the present invention for the preparation of a medicament for the treatment of Alzheimer's disease or other cerebrovascular amyloidosis. The inhibitors of beta-secretase identified by the competitive binding assay of the present invention may be useful for the treatment of neurological disorders and other disorders involving A-beta, APP, and/or A-beta/APP associated macromolecules, and other macromolecules associated with the active center of BACE binding.

The present invention further relates to a method of treating a patient afflicted or having a predisposition for cerebrovascular amyloidosis, comprising administering to the patient a pharmaceutically effective dose of a compound effective to inhibit beta-secretase identified by the assay or the method of the present invention. The condition of cerebrovascular amyloidosis includes Alzheimer's disease.

Having now generally described this invention, the same will become better understood by reference to the specific examples, which are included herein for purpose of illustration only and are not intended to be limiting unless otherwise specified, in connection with the following figures.

### EXAMPLES

Commercially available reagents referred to in the examples were used according to manufacturer's instructions unless otherwise indicated.

### Example 1

### Cloning and Expression of BACE

cDNA encoding the human aspartyl protease BACE and BACE-2 was modified by PCR in the 5′ non coding region to optimize ribosomal recognition by a Kozack sequence and the cloning efficiency by exchanging GC-rich codons and at the 3′ prime end by adding a sequence encoding 6×His residues to enable rapid purification of the recombinant protein. (SEQ ID NO. 1 and SEQ ID NO. 2, respectively). The start ATG is found at position 16 in SEQ ID NO. 1 and 2. Expression in Sf9 insect cells (Glycoconj. J, 1999, 16(2), 109-123) via recombinant baculovirus resulted in higher yields than expression in E. coli, S. pombe or HEK293 cells. Thus the cDNA was cloned into the pFASTBAC1 vector (Life Technologies. Inc.) as a BamHI×XbaI fragment for expression in insect cells and the PCR product was confirmed by sequencing. After recombination into the baculovirus genome the purified viral DNA was transformed into the insect cells. Sf9 cells were cultured at 27° C. in TC100 medium (BioWhittaker) with 5% (v/v) fetal calf serum. Virus stocks were generated with a titer of 1.5×10⁹ pfu/ml. For large scale production of BACE and BACE-2, 24 L fermenters of Sf9 cells were infected with a MOI of 1.

### Example 2

### Purification of Full-Length BACE

50 g (wet weight) of Sf9 cells was suspended in 750 ml of PBS, 2% Triton X-100 and homogenised with a hand-held glass homogeniser. The homogenate was stirred on ice for 30 min and then centrifuged at 100,000×g for 20 min. The supernatant was adjusted to pH 8.0 and loaded on a 2.6×2.5 cm Ni²⁺NTA-Sepharose column (Qiagen, Germany) which had been equilibrated in 50 mM sodium phosphate, 10 mM Tris 100 mM NaCl, 0.1% Triton X-100, pH 8.0. The column was subsequently washed with this buffer and then with 50 mM sodium phosphate pH 7.4, 100 mM NaCl, 0.1% Triton X-100. The column was then eluted with 50 mM sodium phosphate pH 7.4, 100 mM NaCl, 200 mM imidazole, 0.1% Triton X-100 (10 column volumes). Pooled fractions containing full-length BACE were passed over a 5 ml HiTrap Q column (Pharmacia, Switzerland) and the unbound material collected. This material was then diluted 10-fold into 50 mM TrisHCl pH 7.4, 10 mM NaCl, 0.1% Triton and re-loaded onto a second 5 ml HiTrap Q column which had been equilibrated in 50 mM TrisHCl pH 7.4, 10 mM NaCl, 0.1% Triton X-100. The column was washed in this buffer and eluted with a gradient of 50 mM TrisHCl pH 7.4, 1M NaCl, 0.1% Triton X-100 (20 column volumes). Fractions containing BACE were pooled, dialysed against 50 mM TrisCl pH 8.0, 0.1% Triton X-100 and loaded on a Mono S HR5/5 column (Pharmacia, Switzerland). The unbound material, containing BACE, was pooled and stored at 4° C.

### Example 3

### Synthesis of the Peptide Compound A

Continuous-flow solid-phase synthesis was performed on a Pioneer™ Peptide Synthesis System, starting from Tenta Gel S RAM resin (0.25 mmole/g (Rapp Polymere GmbH, Tübingen, Germany) according to the method described by Atherton and Sheppard, Solid Phase Peptide Synthesis: A Practical Approach (IRL Press Oxford 1989). The base-labile Fmoc group was used for α-amino protection. Side chains were protected with the following protection groups: -Asn(Trt) and Glu(OtBu). Commercially available Fmoc-Statine (Neosystem) and Fmoc-3,5-diido-L-tyrosine (Fluka) were used in synthesis. Fmoc-amino acids (2.5 equiv.) were activated with an equivalent amount of O-(1,2-dihydro-2-oxopyrid-1-yl)-N,N,N′,N′-tetramethyluronium tetrafluoroborate (TPTU) and N,N-diisopropylethylamine (Hünig's base). Fmoc deprotection was achieved with 20% piperidine in DMF. Glu(OBut)-Val-Asn(Trt)-Statine-Val-Ala-Glu(OtBu)-Tyr (I₂)-amide Tenta Gel S-resin (0.200 g) was treated with a mixture (5 ml) of 95% TFA, 2.5% H₂O, 2.5% triisopropylsilane for 5 hours. The reaction mixture was concentrated and poured into diethyl ether and the precipitate was collected by filtration and lyophilized from water. The crude peptide was purified by preparative RP-HPLC. There was obtained homogeneous Glu-Val-Asn-Statine-Val-Ala-Glu-Tyr(I₂)—NH₂ (Compound A; 12 mg, MH⁺=1231.3). Other beta-secretase inhibitors were synthesized analogously.

### Example 4

### Tritiation of Compound A

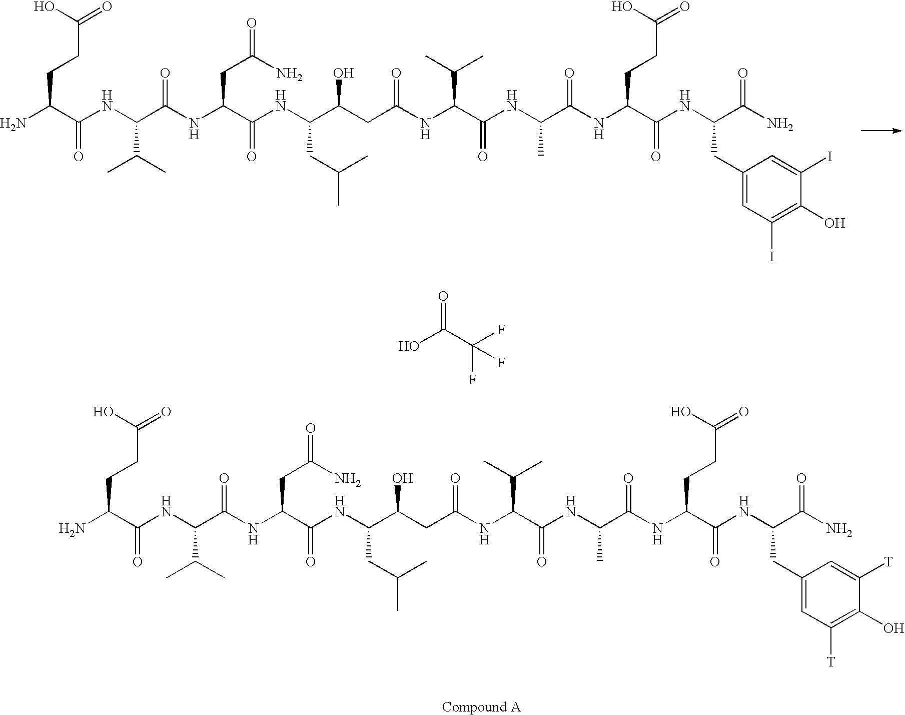

9.3 mg (0.0069 mmoles) of Compound A was dissolved in 1 ml methanol (Merck #1.06009.1000) and 6 drops of water. After addition of 7 μl triethylamine (Fluka puriss #90335) and 4 mg of 10% palladium on charcoal (Degussa Typ101ND) the suspension was stirred in an atmosphere of tritium gas for 1 h. A tritiation apparatus from RC Tritec AG, 9053 Teufen was used (Helv. Chim. Acta, 1985, 68, 1880). After removal of the volatile tritium components the residue was suspended in methanol—water 9/1. After brief sonication the suspension was filtered through Millex-HV 0.45 μm (Cat. No. SL HV013 NL). The filtrate was diluted to 100 ml methanol—water 9/1. The total ³H-activity was 108.54 mCi. and the radiochemical purity was 84% according to HPLC (column Vydac 5μ 4.6×250 mm, flow rate 1 ml/min, solvent A: 95% water—5% acetonitrile—1% TFA; solvent B: 10 mM TFA in water—acetonitrile 3/1; gradient B: 0-40% in 30 min. retention time: 23.27 min; λ=254 nm). The specific activity was 55 Ci/mmole as determined by mass spectrometry. The Millex-HV filter was rinsed several times with water—methanol 9/1, which gave another batch of 66 mCi of the peptide. Purification of this batch by HPLC furnished 43.4 mCi of tritiated Compound A with 100% purity according to HPLC.³H-NMR showed one single peak near 7 ppm thus excluding the presence of any labile tritium.

### Example 5

### FRET Assay for Characterization of Novel Beta-Secretase Inhibitors

All enzyme assays were performed at 20° C. on a FLUOstar (BMG Lab Technologies, D-77656 Offenburg) using 96-well microtiter plates (DYNEX Microfluor 2, Chantilly, Va., USA). The assay volume was 100 μl. Typically, inhibitors dissolved in dimethyl sulfoxide were added in different concentrations into a well followed by buffer and enzyme. The dimethyl sulfoxide concentration was kept below 4%. The enzymatic reaction was started by adding the substrate. pH and buffer conditions under which the experiments were carried out are indicated in the figure and table legends. The progression of the fluorescence increase was measured at λₑₘᵢₛₛᵢₒₙ=520 nm with fluorescence excitation at λ_{excitation}=430 nm. Reaction kinetics were followed periodically during 30 min at various substrate concentrations. The detected signals were converted into moles of substrate hydrolyzed per second. Kinetic data were determined graphically from Lineweaver-Burke plots. It should be noted that data obtained by varying substrate concentration had to be corrected for the effect of excess quenching capacity which is typical of all the FRET substrates used here.

Assays were performed at enzyme concentrations that warranted a linear progression of product formation.

### Example 6

### Competitive Radioligand Binding Assay (RLBA)

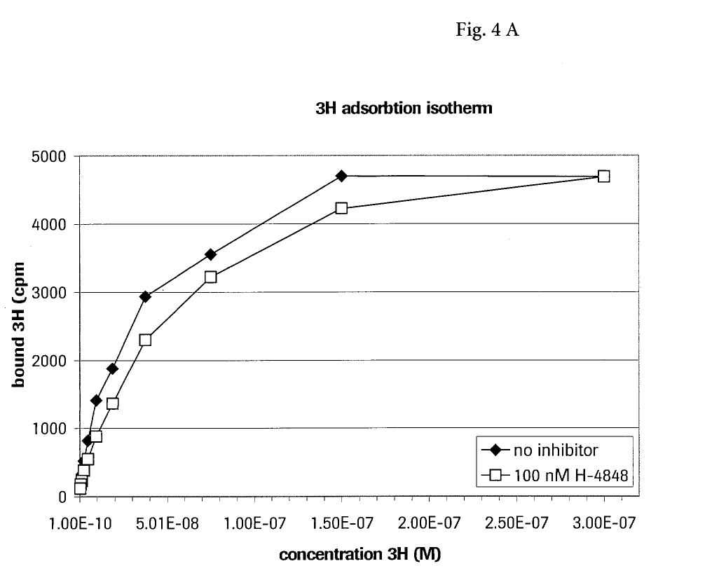

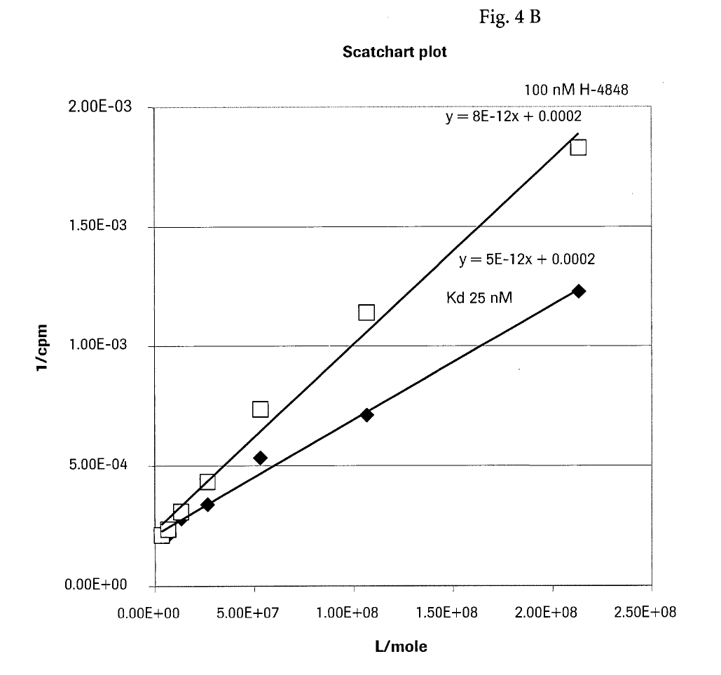

96 well microplates (Optiplate Packard) are coated with purified BACE protein using a concentration of 1 μg/ml in 30 mM sodium citrate buffer adjusted to pH 5.5. The coating is achieved by incubation of 100 μl/well for 1-3 days at 4° C. The plate is then washed with 2×300 μl/well of 10 mM citrate pH 4.1. To each well 100 μl binding buffer (30 mM citrate, 100 mM NaCl, 0.1% BSA, pH 4.1) is dispensed. The test compound (peptide H-4848 in FIGS. 4A and B) is added in 5 μl from a DMSO stock solution or appropriate dilutions. To this the tracer (tritiated Compound A) is added in 10 μl/well from a 10 μCi/ml stock solution in binding buffer. After incubation for 1.5-2 hours in a humid chamber at ambient temperature the plate is washed with 2×300 μl/well water and flipped on a dry towel. Following the addition of 50 μl/well MicroScint20 (Packard) the plate is sealed and vibrated for 5 seconds. The bound radioactivity is counted on a Topcount (Packard). Total binding is typically between 2000 and 10000 cpm/well depending mainly on the purity and concentration of the BACE protein. Non-specific binding as assessed by competition with >1 μM peptide H-4848 (Bachem #H-4848) is typically between 30 and 300 cpm/well. The IC-50 values are calculated by Microsoft Excel FIT.

1. A beta-secretase inhibitor of Formula I:Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

2. The beta-secretase inhibitor according to claim 1 which is used to prepare a a tagged beta-secretase inhibitor.

3. The beta-secretase inhibitor according to claim 1 which comprises a tag.

4. The beta-secretase inhibitor according to claim 3 which is radioactively-tagged at at least one position at P3, P1 or P4′.

5. An assay for identifying beta-secretase inhibitors comprising the steps of
(a) immobilizing beta-secretase protein;
(b) contacting the immobilized beta-secretase protein with a a test compound followed by a tagged beta-secretase inhibitor;
(c) incubating the assay components; and
(d) measuring bound tagged beta-secretase inhibitor.

6. The assay according to claim 5, wherein the beta-secretase is isolated and purified.

7. The assay according to claim 5, wherein the beta-secretase is recombinantly produced and purified.

8. The assay according to claim 5, wherein the beta-secretase is a full-length beta-secretase.

9. The assay according to claim 5, wherein the beta-secretase is selected from the group consisting of BACE and BACE-2.

10. The assay according to claim 5, wherein the tagged beta-secretase inhibitor is a beta-secretase inhibitor labelled with a radioactive tag.

11. The assay according to claim 10, wherein the radioactive tag is tritium.

12. The assay according to claim 5 wherein the beta-secretase inhibitor comprises a beta-secretase inhibitor of Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

13. The assay of claim 12 wherein the beta-secretase inhibitor comprises a tag.

14. The assay according to claim 5, wherein the tagged beta-secretase inhibitor has an inhibitory concentration of about or less than 1 μM.

15. The assay according to claim 5, wherein the components of the assay are incubated in a binding buffer.

16. The assay according to claim 15 wherein the binding buffer contains BSA.

17. The assay according to claim 15 wherein the components of the assay are incubated in binding buffer containing a non-ionic detergent.

18. The assay according to claim 17, wherein the non-ionic detergent is Tween-20.

19. The assay according to claim 5 wherein the components of the assay are incubated in binding buffer optionally containing an ionic detergent.

20. The assay according to claim 19, wherein the ionic detergent is selected from the group comprising cholic acid and deoxycholic acid.

21. A method of screening for compounds capable of inhibiting a beta-secretase activity comprising measuring the binding activities of a tagged beta-secretase inhibitor to a beta-secretase in the presence of a test compound and determining the level of test compound competing with the tagged beta-secretase inhibitor for active-site binding based on the binding activities.

22. The method according to claim 21, wherein the beta-secretase is isolated and purified.

23. The method according to claim 21, wherein the beta-secretase is recombinantly produced and purified.

24. The method according to claim 21, wherein the beta-secretase is a full-length beta-secretase.

25. The method according to claim 21, wherein the beta-secretase is selected from the group consisting of BACE and BACE-2.

26. The method according to claim 21, wherein the tagged beta-secretase inhibitor is a beta-secretase inhibitor labelled with a radioactive tag.

27. The method according to claim 26, wherein the radioactive tag is tritium.

28. The method according to claim 21, wherein the tagged beta-secretase inhibitor is a beta-secretase inhibitor of Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

29. The method of claim 28 wherein the beta-secretase inhibitor comprises a tag.

30. A kit for identifying a beta-secretase inhibitor comprising natural or recombinantly produced beta-secretase polypeptide and a tagged beta-secretase inhibitor.

31. The kit according to claim 30, wherein the tagged beta-secretase inhibitor carries a radioactive tag.

32. The kit according to claim 30, wherein the tagged beta-secretase inhibitor comprises a tritium tag.

33. A kit for use in an assay to identify beta-secretase activity, wherein the assay comprises the steps of
(a) immobilizing beta-secretase protein;
(b) contacting the immobilized beta-secretase protein with a a test compound followed by a tagged beta-secretase inhibitor;
(c) incubating the assay components; and
(d) measuring bound tagged beta-secretase inhibitor.

34. A kit for screening compounds in accordance to a method capable of inhibiting a beta-secretase activity, the method comprises measuring the binding activities of a tagged beta-secretase inhibitor to a beta-secretase in the presence of a test compound and determining the level of test compound competing with the tagged beta-secretase inhibitor for active-site binding based on the binding activities.

35. An inhibitor of beta-secretase identified by an assay wherein the assay comprises the steps of
(a) immobilizing beta-secretase protein;
(b) contacting the immobilized beta-secretase protein with a a test compound followed by a tagged beta-secretase inhibitor;
(c) incubating the assay components; and
(d) measuring bound tagged beta-secretase inhibitor.

36. An inhibitor of beta-secretase identified by a method of screening compounds capable of inhibiting a beta-secretase activity, the method comprises measuring the binding activities of a tagged beta-secretase inhibitor to a beta-secretase in the presence of a test compound and determining the level of test compound competing with the tagged beta-secretase inhibitor for active-site binding based on the binding activities.

37. A pharmaceutical composition comprising a pharmaceutically acceptable carrier and a therapeutically effective amount of a beta-secretase inhibitor identified by an assay wherein the assay comprises the steps of
(a) immobilizing beta-secretase protein;
(b) contacting the immobilized beta-secretase protein with a test compound followed by a tagged beta-secretase inhibitor;
(c) incubating the assay components; and
(d) measuring bound tagged beta-secretase inhibitor.

38. A pharmaceutical composition comprising a pharmaceutically acceptable carrier and a therapeutically effective amount of a beta-secretase inhibitor which is identified by a method of screening compounds capable of inhibiting a beta-secretase activity comprising measuring the binding activities of a tagged beta-secretase inhibitor to a beta-secretase in the presence of a test compound and determining the level of test compound competing with the tagged beta-secretase inhibitor for active-site binding based on the binding activities.

39. The pharmaceutical composition according to claim 37 comprising a beta-secretase inhibitor of Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

40. The pharmaceutical composition according to claim 38 comprising a beta-secretase inhibitor of Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

41. A method of treating a patient afflicted or having a predisposition for cerebrovascular amyloidosis comprising administering to the patient a pharmaceutically effective amount of a compound comprising a beta-secretase inhibitor identified by an assay for identifying beta-secretase inhibitors, the assay comprising the steps of
(a) immobilizing beta-secretase protein;
(b) contacting the immobilized beta-secretase protein with a test compound followed by a tagged beta-secretase inhibitor;
(c) incubating the assay components; and
(d) measuring bound tagged beta-secretase inhibitor.

42. A method of treating a patient afflicted or having a predisposition for cerebrovascular amyloidosis comprising administering to the patient a pharmaceutically effective amount of a compound comprising a beta-secretase inhibitor identified by a method for screening compounds capable of inhibiting a beta-secretase activity, the method comprises measuring the binding activities of a tagged beta-secretase inhibitor to a beta-secretase in the presence of a test compound and determining the level of test compound competing with the tagged beta-secretase inhibitor for active-site binding based on the binding activities.

43. The method according to claim 41 wherein the cerebrovascular amyloidosis is Alzheimer's disease.

44. The method according to claim 42 wherein the cerebrovascular amyloidosis is Alzheimer's disease.

45. The method according to claim 41 wherein the beta-secretase inhibitor has the Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.

46. The method according to claim 42 wherein the beta-secretase inhibitor has the Formula I: Y-P4-P3-P2-P1-P1′-P2′-P3′-P4′-W,
wherein P1 is defined as Leustatin, Chastatin or Tyrstatin;
P2 is defined as Asn;
P3 is defined as Val, Cpe, Che or Cha;
P4 is defined as Glu;
P1′ is defined as Val;
P2′ is defined as Ala;
P3′ is defined as Glu;
P4′ is defined as Tyr, Cha, Phe(I) or Tyr(I2);
Y is defined as 0 to 10 amino acid residues, and
W is defined as 0 to 10 amino acid residues,
or a combination thereof.
